(19) United States
(12) Patent Application Publication
(10) Pub. No.: US 2023/0406786 A1
(43) Pub. Date: Dec. 21, 2023

(54) Process and Plant for Producing One or More Hydrocarbons

(71) Applicant: LINDE GMBH, Pullach (DE)

(72) Inventors: Andreas Meiswinkel, Rimsting (DE); Hans-Jorg Zander, München (DE)

(21) Appl. No.: 18/251,732

(22) PCT Filed: Nov. 4, 2021
(86) PCT No.: PCT/EP2021/080669

§ 371 Date: May 4, 2023

(30) Foreign Application Priority Data
Nov. 6, 2020 (DE) 10 2020 129 303.8

(51) Int. Cl. C07C 1/04; C07C 1/12

(52) U.S. Cl. CPC C07C 1/0475; C07C 1/12

(57) ABSTRACT

A method for producing one or more hydrocarbons includes feeding a process gas stream into a reactor arrangement. The process gas stream includes carbon dioxide and/or carbon monoxide with hydrogen, which are converted at least in part in a first reaction step into one or more oxygenates, which are converted at least in part in a second reaction step into the one or more hydrocarbons. The reactor arrangement has one or more reactors, which comprise a first reaction zone and a second reaction zone arranged downstream of the first reaction zone. The first reaction zone and the second reaction zone are equipped with catalysts in such a way that the first and second reaction steps are catalyzed in the first reaction zone and that the second reaction step is catalyzed in the second reaction zone. In the second reaction zone, the first reaction step is generally not catalyzed.

### CROSS-REFERENCE TO RELATED APPLICATIONS

This application is the national phase of, and claims priority to, International Application No. PCT/EP2021/080669, filed 4 Nov. 2021, which claims priority to German Application No. DE 10 2020 129 303.8, filed 6 Nov. 2020.

### FIELD OF THE INVENTION

The invention relates to a method for producing one or more hydrocarbons, in particular ethylene and/or propylene, and to a corresponding system.

### BACKGROUND

Ethylene and propylene are essential building blocks in petrochemistry. Further continuous growth is expected for both products. The annual need in 2016 was 150 million t/a for ethylene (capacity of 170 million t/a) and 100 million t/a for propylene (capacity of 120 million t/a). In particular, an increasing need for propylene is predicted (“propylene gap”), which requires the provision of corresponding selective methods.

In principle, changes in the raw material supply affect the petrochemical value chain and cause an increased demand for new production routes for the mentioned olefins and further hydrocarbons. At the same time, the carbon dioxide footprint of corresponding methods is to be reduced and carbon dioxide emissions are to be reduced or eliminated as far as possible. The aim of academic and industrial research activities, and also of the invention, is therefore to identify alternative production routes for hydrocarbons, which also take into account energy and environmental aspects.

The invention therefore aims at an improved method for producing hydrocarbons from carbon dioxide. In addition to the general design, the production of paraffins and particularly preferably of olefins is in particular the aim of the invention. The invention is in particular aimed at the production of paraffins and olefins having two to eight carbon atoms but in particular two and three carbon atoms. The following statements are accordingly partially strongly geared toward obtaining ethylene and propylene. As by-products, aromatics may also arise. However, the invention is not limited to these hydrocarbons.

Existing methods for selectively producing olefins are explained in more detail below. In these methods, significant amounts of carbon dioxide are usually released, either from firing or energy supply in endothermic processes, or else as by-product in oxidative processes. Further by-products are often also carbon monoxide and hydrogen, which have hitherto likewise only been fed to a limited extent to a material utilization and are frequently used for firing, for example.

Current fields of research, as are also explained in more detail below, are aimed at the conversion of carbon dioxide with hydrogen (optionally also in the presence of carbon monoxide) into hydrocarbons. The focus in this respect is in particular on products having two to four carbon atoms (both paraffins and olefins), in particular propylene.

The invention in particular proceeds from methods which currently lead to hydrocarbons via methanol and/or dimethyl ether as an (isolated) intermediate stage. This relates to the synthesis of methanol and/or dimethyl ether from synthesis gas, which is explained in more detail below, followed by the methanol-to-olefin or methanol-to-propylene methods, likewise explained in more detail below. According to the prior art, these methods are performed in two stages, i.e., sequentially in separate reaction steps and via different catalysts.

### SUMMARY

An object of the invention is to improve corresponding methods and, in particular, to design them more advantageously with regard to their energy consumption and/or carbon dioxide footprint.

According to an embodiment of the invention, a method for producing one or more hydrocarbons includes feeding a process gas stream to a reactor arrangement, the process gas stream comprising carbon dioxide and/or carbon monoxide with hydrogen. The process gas stream is converted, within the reactor arrangement, at least in part in a first reaction step into one or more oxygenates, which pass into the process gas stream. The one or more oxygenates in the process gas stream are converted, within the reactor arrangement, at least in part in a second reaction step into one or more hydrocarbons, which pass into the process gas stream. The process gas stream is conducted in a flow direction through the reactor arrangement. The reactor arrangement has one or more reactors, which comprise a first reaction zone and a second reaction zone, the second reaction zone being arranged downstream of the first reaction zone in the flow direction. The first reaction zone and the second reaction zone are equipped with catalysts in such a way that the first and second reaction steps are catalyzed in the first reaction zone; the second reaction step is catalyzed in the second reaction zone; and in the second reaction zone, the first reaction step is not catalyzed or is catalyzed to a lesser extent than in the first reaction zone.

### BRIEF DESCRIPTION OF THE DRAWINGS

FIG. 1 shows a method according to an embodiment of the invention.

FIGS. 2A and 2B illustrate advantages of an embodiment of the invention over the prior art by means of conversion diagrams.

### WRITTEN DESCRIPTION

The invention proceeds from methods which currently lead to hydrocarbons via methanol and/or dimethyl ether as an (isolated) intermediate stage and which are explained in detail below. However, prior to the explanation of the specific advantages and features of embodiments of the present invention, reference is to be made again, by way of comparison, to further methods that may be used in connection with the present invention, in the further context thereof, and as an alternative to the present invention.

As mentioned, in the chemical industry, the desire to reduce the carbon dioxide emission of synthesis methods or corresponding systems exists. Accumulating carbon dioxide is advantageously to be used materially. For illustrative purposes only, reference is made in this connection to the relevant technical literature cited at the end, here in particular references [1] to [6].

Relevant synthesis methods for the production of olefins, such as ethylene and propylene, comprise, for example, steam cracking, wherein inputs, such as ethane, propane, so-called liquefied petroleum gas (LPG) or naphtha, may be used and, in particular for the production of propylene, fluid catalytic cracking using a corresponding catalyst. Recent trends in olefin production are specified in [7], for example.

Alternative technologies for producing ethylene comprise the generally known oxidative dehydrogenation of ethane (ODH-E), in which acetic acid arises as a co-product, and the likewise known oxidative coupling of methane. Methods for producing propylene include, for example, the established propane dehydrogenation and olefin metathesis, which requires 2-butene as input.

Modified Fischer-Tropsch methods, which are optimized to form a maximum yield of light olefins (“Fischer-Tropsch-to-olefins,” FTTO), and Fischer-Tropsch methods, which are combined with a reverse water-gas shift (RWGS) so that carbon dioxide can also be incorporated, can be mentioned here. Reference is in particular made to the technical literature such as [8] and [9] regarding FTTO and [10] to [12] regarding the combination of Fischer-Tropsch methods with RWGS.

In addition, there is a large number of further developments and publications which, inter alia, are also based on the use of dimethyl ether (in turn as an isolated intermediate stage) as input and/or attempt to achieve a particularly advantageous product distribution. Again, reference can be made to the relevant technical literature, such as [13] to [16].

The production of oxygenates, such as methanol and dimethyl ether from synthesis gas, is already largely established prior art, as is basically also the combination with so-called methanol-to-olefin or methanol-to-propylene methods (MTO, MTP), which, irrespective of their designation, can also convert other oxygenates, such as dimethyl ether, in a comparable manner. Corresponding method combinations represent a further route to the production of olefins. Regarding the background and technical execution, reference is also made here to the relevant technical literature, see [17] to [21], for example.

Technologies for providing synthesis gas as used in such methods are likewise described extensively in the technical literature, see [22] to [26], for example. They include, inter alia, partial oxidation and different reforming methods. So-called dry reforming (also referred to as carbon dioxide reforming) with downstream shift for adjusting the hydrogen/carbon monoxide ratio is also established.

The mentioned methanol-to-olefin or methanol-to-propylene methods conventionally proceed from methanol and/or dimethyl ether as an isolated intermediate stage, which can be produced by converting synthesis gas (for example produced from methane but also from carbon, naphtha, etc.). In particular, olefins, such as ethylene and propylene, are desired as the preferred target product. The catalysts used are Lewis-acidic materials, in particular zeolites or zeolite-like materials. By selecting the catalyst material and the exact reaction conditions, the product spectrum can be adapted, in particular with respect to the type of products and their relative distribution. In general, zeolites (base type ZSM-5, average porosity) and silicoaluminophosphates (SAPO, in particular SAPO-34, low porosity) in particular have nowadays been found to be best suited for technical application.

Methanol-to-olefin or methanol-to-propylene methods are commercially established and are performed, for example, in the form of a methanol-to-propylene method based on a zeolite catalyst and a fixed-bed reactor (one or two reactor systems with additional reserve reactor are common) and in the form of a methanol-to-olefin method with a catalyst based on SAPO-34. In the former, a main advantage is to exist in the simple expansion capability of the fixed-bed reactor (parallelization) and the significantly lower investment costs, wherein essentially propylene is desired as the target product but significant amounts of heavier fractions also additionally arise. In the latter, the advantage in particular is that narrower microcrystalline pores are present in SAPO-34 than in ZSM-5 and better control of the acidity can be achieved. In the latter method, crude methanol is typically used and the reactor system used comprises two fluidized bed reactors (each for the actual reaction and for continuous catalyst regeneration), wherein the process is controlled by the temperature at 350 to 525° C. (preferably 350° C.), the pressure at 1 to 3 bar, the dwell time, and the regeneration cycle.

Developments or optimizations include in particular the combination with an additional catalytic cracking step (olefins cracking process, OCP) for a recycle stream in order to increase olefin selectivity.

For example, the technical literature also describes a combination of reforming and so-called Fischer-Tropsch-to-olefin methods via methanol to olefins. Furthermore, approaches in order to combine a methanol synthesis from synthesis gas and methanol-to-olefin or methanol-to-propylene methods in one step are also already found therein. These approaches are essentially based on a combination of Zr-Zn oxides (for methanol synthesis) and H-SAPO-34 as methanol-to-olefin or methanol-to-propylene catalyst.

There are also already developments for the hydrogenation of carbon dioxide via bifunctional catalysts (in this respect, for example, review article [27]).

Generally, however, corresponding systems also seem to be suitable for the corresponding conversion of carbon monoxide or any mixtures of carbon monoxide and carbon dioxide into hydrocarbons and in particular into olefins, such as ethylene and/or propylene.

The bifunctional catalysts catalyze two reaction steps, namely a conversion of the components of synthesis gas into one or more oxygenates, such as methanol and/or dimethyl ether as intermediate(s), and the further conversion of the intermediate(s) into the desired target compound in the form of the one or more hydrocarbons. A bifunctional catalyst used in this case combines typically used methanol catalysts with acidic zeolite structures, which catalyze the subsequent reaction.

Alternatively, the two reaction steps running parallel can, however, also take place by a combination of two or more suitable catalysts, each of which preferably catalyzes the corresponding reaction steps on its own, as physical mixture in a catalyst bed. The corresponding catalysts can be the basically known, aforementioned catalysts for methanol or dimethyl ether synthesis or for further conversion.

As viewed from the outside, the overall reaction in both variants takes place as a single-stage reaction, which however comprises two individual reactions or reaction steps.

For propylene as target product, the following individual reactions result, for example, when starting from methanol:

CO+2H₂ [symbol] CH₃OH ΔH=−91 kJ/mol (1)

CO2+3H₂ [symbol] CH₃OH+H₂O ΔH=−48 kJ/mol (2)

3CH₃OH [symbol] C₃H₆+3H₂O ΔH=−104 kJ/mol (3)

As a side reaction or “overhydrogenation” to propane, the following can be observed:

3CH₃OH+H₂→C₃H₈+3H₂O ΔH=−227 kJ/mol (4)

Traditional two-stage reaction systems are to be regarded rather as disadvantageous due to thermodynamic conditions compared to a single-stage reaction as described here. Frequently, various olefins, such as ethylene and propylene as well as different amounts of paraffins (ethane, propane) and also heavier hydrocarbons, arise in corresponding methods. Aromatics can likewise arise as by-products.

Examples of bifunctional catalysts used for the conversion of carbon dioxide are given in the following Table 1.

TABLE 1

| Citation | Catalyst and comments |
|---|---|
| [28] | In₂O₃/ZrO₂ + SAPO-34 |
| [29] | ZnGa₂O₄ + SAPO-34 |
| [30] | Zn@SiO₂@SAPO-34 |
| [31] | ZnO—ZrO₂ ″solid solution″ on Zn-modified |
| | SAPO-34-zeolite |
| [32] | Composite catalyst: Cu—Zn—Al oxide and modified |
| | HB zeolite (modification with 1,4-bis(hydroxy- |
| | dimethylsilyl)benzene) for the generation of |
| | a hydrophobic surface. |
| [33] | Zr-modified Cu—Zn catalyst with Pd-modified β-zeolite. |
| [34] | In—Zr oxide/SAPO-34. A further catalyst overview |
| | is found in chapter 3 of the reference. |
| [35] | Different bifunctional catalysts, e.g., In₂O₃/HZSM-5 |
| | (2/1) with very high formation of C5+ or Zn—Zr/ |
| | HZSM-5 (2/1) with high formation of C5+ and |
| | considerable C₂—C₄ formation. |
| [36] | Primarily CuZnOZrO₂ and zeolites (MFI or SAPO). |
| [37] | Cu—ZnO—Al₂O₃ and HZSM-5. |
| [38] | First stage: Cu—Zn—Al oxide catalyst: RWGS |
| | reaction to form carbon monoxide, Second stage: |
| | Cu—Zn—Al oxide and HB to form MeOH and catalysis |
| | of the MTG reaction (methanol-to-gasoline). Water |
| | removal between the two stages. Main products: light |
| | paraffins, essentially propane and i-butane. |
| [39] | First stage: hybrid catalyst Cu—Zn—Zr—Al/Pd— |
| | B-zeolite, Second stage: hybrid catalyst or Pd-β zeolite. |
| | Goal: production of LPG (C3 and C4 |
| | paraffins), two stages with intercooler (0° C.). |

The invention proposes a combination of the two mentioned reaction steps in one reactor or in a plurality of reactors with a suitable catalyst (wherein one or both of the reaction steps according to the above equations (1) and (2) are considered as a first reaction step and the reaction step 3 according to the above equation (3) is considered as second reaction step.

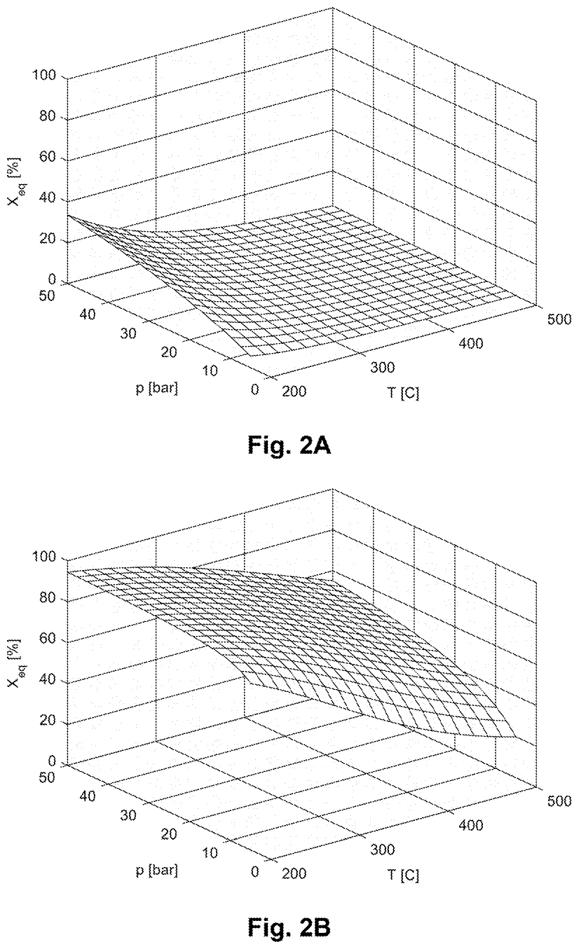

As illustrated with reference to FIGS. 2A and 2B, the use of the invention results in a significantly higher yield than in a sequential performance.

The reactions involved are in principle thermodynamically limited equilibrium reactions. It follows therefrom that in principle, all species involved (starting materials, such as carbon monoxide, carbon dioxide and hydrogen, intermediates, such as methanol and/or dimethyl ether, and hydrocarbons, such as in particular ethylene and propylene as products) are contained at any time in the reaction matrix. In addition to the reaction kinetics, the corresponding fraction is in particular determined by the thermodynamic equilibrium position. In particular at the reactor outlet, the composition should come closer and closer to this thermodynamic equilibrium.

If the conversion of carbon dioxide into propylene starting from a stoichiometric mixture is considered, it comprises the reactions according to the above equations (1) and (3). The diagram according to FIG. 2A illustrates the conversion X_{eq} in percent for the case that the reactions are brought into equilibrium one after the other, on the vertical axis over a pressure p in bar on the horizontal axis shown on the left and a temperature in ° C. on the horizontal axis shown on the right. The diagram according to FIG. 2B, on the other hand, with identical axes as in FIG. 2A, illustrates the conversion X_{eq} for the case that both reactions are brought into equilibrium simultaneously.

A combination of both steps (here the methanol synthesis combined with the conversion into propylene) in a reactor with a suitable bifunctional catalyst or a mixture of suitable catalysts for both reaction steps accordingly results in a significantly higher yield than in a sequential performance of the two reactions.

Overall, the invention proposes a method in which carbon dioxide and/or carbon monoxide with hydrogen in a process gas stream, which is fed to a reactor, are converted at least in part in a first reaction step into one or more oxygenates, which pass into the process gas stream, and in which the one or more oxygenates in the process gas stream are converted at least in part in a second reaction step into the one or more hydrocarbons, which pass into the process gas stream, wherein the process gas stream is conducted through a reactor arrangement in a flow direction. A “reactor arrangement” is to be understood as an arrangement which has, as minimum equipment, one or more reactors and components required in the operation thereof.

Within the scope of the invention, the reactor arrangement comprises one or more reactors, which have a first and a second reaction zone, wherein the second reaction zone is arranged downstream of the first reaction zone in the flow direction, and wherein the first reaction zone and the second reaction zone are equipped with catalysts in such a way that the first and second reaction steps are catalyzed in the first reaction zone, that the second reaction step is catalyzed in the second reaction zone, and that in the second reaction zone, the first reaction step is not catalyzed or is catalyzed to a lesser extent than in the first reaction zone. The term “lesser extent” is understood to mean, for example, a relative conversion of an amount of substance of less than 10%, in particular 5% and in particular 2%, of the amount of substance converted in the first reaction step in the first reaction zone.

In other words, within the scope of the invention, the first and second reaction zones can be located in a reactor of the reactor arrangement or two reactors can be provided which have the two reaction zones one behind the other in the aforementioned arrangement. For reasons of simplification only, “a” reactor is referred to below in this respect.

In other words, within the scope of the invention, a second reaction zone in which the one or more oxygenates, such as methanol and/or dimethyl ether, are predominantly or exclusively converted, but not carbon dioxide, carbon monoxide and hydrogen, which however continue to be contained in the process gas stream, follows a first reaction zone in which, in addition to the conversion of the one or more oxygenates, the conversion of carbon dioxide, carbon monoxide and hydrogen into the oxygenates also takes place. In this way, the process gas is depleted of the one or more oxygenates in the second reaction zone, which also facilitates the subsequent processing, as explained in more detail below.

Within the scope of the invention, the first reaction zone can be equipped with one or more first catalysts, which catalyze the first reaction step, and also with one or more second catalysts, which catalyze the second reaction step, for example in the form of a physical mixture. Alternatively, it is also possible to equip the first reaction zone with one or more bifunctional catalysts, which catalyze the first and the second reaction step. In both cases, the advantages according to the invention are achieved.

In contrast to the two-stage embodiment, i.e., the sequential sequence of oxygenate synthesis and further conversion into hydrocarbons, a bifunctional catalyst without the use of the measures proposed according to the invention or the mere use of two catalysts simultaneously, i.e., in one catalyst bed, is also disadvantageous for the technical application. Since, as mentioned, complete conversion of carbon monoxide and carbon dioxide cannot be achieved in technically relevant systems, the one or more oxygenates, which are thus contained in significant amounts (at least in the percent range) in the outlet stream of a corresponding reactor, also continue to be formed continuously according to the thermodynamic equilibrium as a result of such a bifunctional catalyst or a corresponding mixture. In this case, the one or more oxygenates pass together with reaction water into a condensate from which the condensates, in particular methanol, can however be separated off and fed to a use only in a comparatively complex manner. An undesired by-product thus unavoidably arises, which can hardly be separated off or used in a cost-effective manner. That is to say, this by-product is thus generally fed together with the condensate to a wastewater conditioning unit and is thus lost from the value chain. For example, methanol can be degraded by suitable bacteria in a biological wastewater treatment, wherein in turn an emission of carbon dioxide occurs. Although this degradation of methanol in biological sewage treatment systems is in principle technically readily possible, this again means corresponding additional outlay (due to the required capacity of the biological treatment). By using the invention, a separation or subsequent wastewater treatment is no longer required or a lower capacity is sufficient in this case since the one or more oxygenates are converted in the second reaction zone.

In other words, according to the invention, the content of oxygenate(s) at the reactor outlet can be effectively minimized and the process efficiency can thus be significantly increased in the direction of the target products. Components, such as carbon monoxide, carbon dioxide and hydrogen, that continue to be contained may then optionally be relatively easily separated from the target products and recycled.

In a further embodiment of the invention, upstream of the first reaction zone according to the invention, one or more further reaction zones can also be arranged, which contain suitable catalysts which in particular catalyze a water-gas shift reaction and/or the formation of methanol and/or dimethyl ether as an intermediate. In these zones, the further conversion into hydrocarbons according to the above definition is not catalyzed or is catalyzed only to a “lesser extent” than in the first reaction zone.

For the reaction management of heterogeneous catalysts, various reactor types are in principle suitable. Fixed-bed reactors, such as described in [40] and [41], are particularly advantageous since they are structurally comparatively simple to realize. In particular, the reactor used within the scope of the invention is therefore designed as a fixed-bed reactor which has fixed catalyst beds in the first reaction zone and in the second reaction zone, which catalyst beds contain the respective catalysts as fixed bed catalysts. The same also applies if a plurality of reactors is used or contained in a corresponding reactor arrangement. A “catalyst bed” typically has a supported catalyst in a suitable holding structure.

Within the scope of the present invention, tube bundle reactors with suitable cooling medium, in particular a salt melt, can be used in particular. In this case, the cooling can take place in cocurrent or countercurrent flow with the process gas stream, and different cooling/heating zones can also be provided within the scope of the invention if required. The reaction zones provided according to the invention are formed by separate catalyst beds arranged in parallel in the reaction tubes or such catalyst beds together in each case form the reaction zones. It is understood that if it is stated in a simplified manner here that “a process gas stream” is conducted through the reactor or a corresponding reactor arrangement, this relates, in the case of a tube bundle reactor, to a number of partial streams corresponding to the number of reaction tubes.

Adiabatic fixed-bed reactors are known, which may optionally also be designed with intercoolers in a multi-stage design. In addition, heated or cooled reactors, which are typically designed as tube bundle reactors, become known in particular for strongly endothermic or exothermic reactions. In particular, systems with a phase change (e.g., water/steam or other evaporating liquids), thermal oils or, in particular at higher temperatures, also salt melts are used as the cooling/heating medium. In this case, the temperature control can take place in cocurrent or countercurrent flow, and different cooling/heating zones are also structurally provided in newer embodiments.

The use of multi-layer catalyst beds or beds in a fixed-bed reactor is also known. In order to increase the overall yield with only minimal losses of commercial product selectivity, multi-layer catalyst beds can be used in conventional processes, optionally with activity increasing along the flow direction in the reactor.

DE 10 2005 004 926 A1 describes, for example, a catalyst system for catalytic gas phase reactions, which is characterized by increasing catalyst activity in the flow direction. However, this increase in activity is achieved exclusively by mixtures of differently active catalysts, which, however, in principle catalyze the same reaction. The methods mentioned are in particular the production of phthalic anhydride, ethylene dichloride, cyclohexanone, maleic anhydride and acrylic acid. A continuous gradient, and not the use of different, defined zones, is expressly proposed.

Relating to oxidative dehydrogenation, EP 3 587 383 A1 also uses a reactor that has a plurality of reaction zones with one catalyst bed each. The plurality of reaction zones can in particular be formed as a layered structure of a plurality of catalyst beds or as reaction zones which are separated from one another and each have one catalyst bed. A formation of corresponding reaction zones in the form of multi-layer catalyst beds, which in this case form a plurality of catalyst beds, is also listed. The catalyst loading and/or catalyst activity is adjusted in particular by different degrees of dilution by means of inert material, but the active catalyst material in the different reaction zones is identical and thus, in principle, catalyzes the same reactions.

Within the scope of the invention, the first reaction zone advantageously has a plurality of catalyst beds which are arranged one behind the other in the flow direction and have a plurality of different catalysts or a catalyst with different activities. This may also relate to a bifunctional catalyst, as can be provided in the first reaction zone. Mixtures of catalysts in different mixing ratios may also be provided. In the method according to the invention, a plurality of catalyst-free inert zones are advantageously furthermore formed in the flow direction. They may, for example, be located upstream of the first and downstream of the second reaction zone. Further inert beds may also be arranged between the reaction beds and/or individual catalyst beds in order to achieve better heat dissipation or temperature control, for example.

Within the scope of the invention, catalysts generally known for the respective reaction system come into consideration without restriction. Reference is made to the above explanations, in particular in connection with Table 1. The invention is characterized less by the types of catalysts used but by the reactions catalyzed by them and by the order and specific manner in which the catalysts used are arranged.

The method according to the invention can be performed at a pressure level of 10 to 100 bar, in particular 12 to 50 bar, more particularly 15 to 35 bar, and at a temperature level of 150 to 580° C., in particular 200 to 450° C., more particularly 250 to 400° C.

The method according to the invention is suitable for the conversion of carbon dioxide and carbon monoxide and gas streams with any mixtures of these two components. In this respect, hydrogen is to be provided in a suitable stoichiometric amount in the reaction feed.

In order to determine the required hydrogen fraction in corresponding reactions, the characteristic number S_{N}, the so-called stoichiometric module, is helpful and customary. It is determined from the amount-of-substance fractions x of carbon monoxide, carbon dioxide and hydrogen as follows:

S_{N}=(xH₂−xCO₂)/(xCO+xCO₂) (5)

The following equations 6 and 7 describe the idealized synthesis of ethylene. Here, SN=2 always applies irrespective of the actual ratio of carbon monoxide to carbon dioxide.

2CO+4H₂→C₂H₄+2H₂O (6)

2CO₂+6H₂→C₂H₄+4H₂O (7)

The following equations 8 and 9 describe the idealized synthesis of propylene, where SN=2 likewise always applies irrespective of the actual ratio of carbon monoxide to carbon dioxide.

3CO+6H₂→C₃H₆+3H₂O (8)

3CO₂+9H₂→C₃H₆+6H₂O (9)

In the case of ethylene and/or propylene as the target product, the proposed method thus preferably uses a feed composition to which at least SN=2 applies (under idealized consideration and without taking into account side reactions). Due to side reactions, adaptation is necessary in reality so that the aforementioned reaction equilibria are advantageous. However, on the other hand, a positive effect of hydrogen excess on the deactivation and coking behavior of the catalyst can usually also be observed.

A limitation of SN upward is likewise advantageous in order to limit the separation and recycling outlay for hydrogen and, on the other hand, to avoid an overreaction of olefins to the corresponding paraffins (“hydrogenation”).

Within the scope of the invention, the process gas stream of the reactor arrangement used in the invention is therefore advantageously fed with a stoichiometric module of 1.5 to 10, in particular 2 to 4.

As mentioned several times, within the scope of the invention, the one or more oxygenates are, in particular, methanol and/or dimethyl ether and the one or more hydrocarbons are, in particular, ethylene and propylene. However, the invention is also, in principle, suitable for other methods of carbon monoxide and/or carbon dioxide hydrogenation, i.e., in particular also the production of higher hydrocarbons having four and more carbon atoms.

Within the scope of the invention, at the inlet of the reactor arrangement, the process gas stream can also have further components, in particular methane and/or higher hydrocarbons, in addition to the mentioned components hydrogen, carbon dioxide and/or carbon monoxide.

After passing through the reactor arrangement, a separation in particular of the hydrocarbons can be carried out, wherein a remaining residue can at least partially be returned to the inlet of the reactor arrangement in order to maximize the overall yield of the method.

The system according to the invention for producing one or more hydrocarbons, in particular ethylene and/or propylene, is configured to convert carbon dioxide and/or carbon monoxide with hydrogen in a process gas stream, which is fed to a reactor arrangement, at least in part in a first reaction step into one or more oxygenates, which pass into the process gas stream, and to convert the one or more oxygenates in the process gas stream at least in part in a second reaction step into the one or more hydrocarbons, which pass into the process gas stream.

According to the invention, the system is configured to conduct the process gas stream in a flow direction through the reactor arrangement, wherein the reactor arrangement has one or more reactors, which comprise a first reaction zone and a second reaction zone, wherein the second reaction zone is arranged downstream of the first reaction zone in the flow direction, and wherein the first reaction zone and the second reaction zone are equipped with catalysts in such a way that the first and second reaction steps are catalyzed in the first reaction zone, that the second reaction step is catalyzed in the second reaction zone, and that in the second reaction zone, the first reaction step is not catalyzed or is catalyzed to a lesser extent than in the first reaction zone.

Regarding features and advantages of a corresponding system and its embodiments, which can in particular be configured for performing a method, as was explained above in different embodiments, reference is expressly made to the above explanations with respect to the method according to the invention and its embodiments.

Again, in summary, the invention achieves a particularly efficient conversion of carbon monoxide and/or carbon dioxide with hydrogen into valuable products. In this case, the conversion or the yield is maximized compared to conventional methods. An integrated method management without isolation of the intermediate or the intermediates is achieved. Nevertheless, there is a minimization of intermediates such as methanol and/or dimethyl ether as (unused) by-products in the outlet stream. The process efficiency toward the desired target products (in particular ethylene and/or propylene) is increased. No complex methanol recovery is required, or the requirements for wastewater treatment (biology) are minimized. Overall, a reduction in production and operating costs results.

The invention is described below with reference to the accompanying drawings, which illustrate an embodiment of the invention and its advantages.

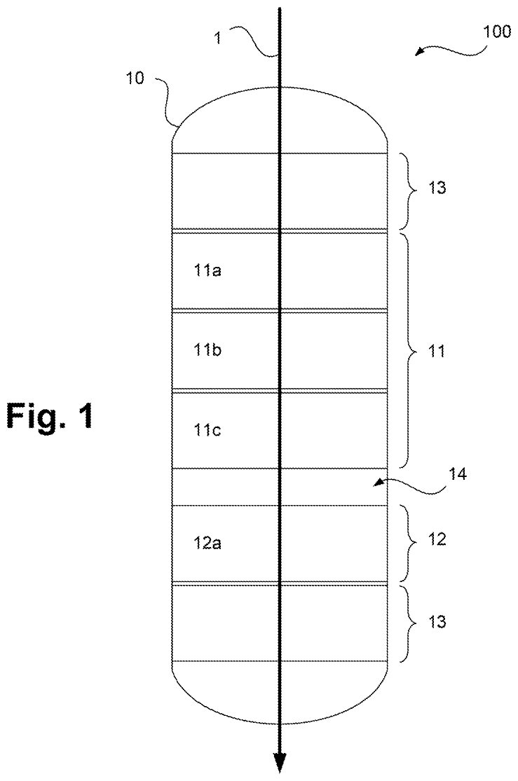

In FIG. 1, a method according to a particularly preferred embodiment of the invention is illustrated in the form of a schematic flow chart and is denoted as a whole by 100. The following explanations relate to a system according to one embodiment of the invention in the same way.

In the method 100, carbon dioxide and/or carbon monoxide with hydrogen in a process gas stream 1, which is fed to a reactor 10, are converted at least in part in a first reaction step into one or more oxygenates, which pass into the process gas stream 1, and the one or more oxygenates in the process gas stream 1 are converted at least in part in a second reaction step into the one or more hydrocarbons, which pass into the process gas stream 1. As mentioned, instead of a reactor 10, an arrangement with a plurality of reactors may also be used within the scope of the invention.

As illustrated by a corresponding arrow, the process gas stream 1 is conducted through the reactor 10 in a flow direction, wherein the reactor 10 has a first reaction zone 11 and a second reaction zone 12, wherein the second reaction zone 12 is arranged downstream of the first reaction zone 11 in the flow direction, wherein the first reaction zone 11 in the example shown has one or more bifunctional catalysts catalyzing the first and second reaction steps, and wherein the second reaction zone 12 has one or more catalysts predominantly or exclusively catalyzing the second reaction step. For further embodiments, reference is made to the explanations above. Instead of the one or more bifunctional catalysts, it is also possible, for example, to use a mixture of a plurality of catalysts, as mentioned. For the sake of simplicity, a “bifunctional” catalyst in the first reaction zone and a “monofunctional” catalyst in the second reaction zone are described within the scope of the invention.

A fixed-bed reactor is used as reactor 10, which in the first reaction zone 11 has a plurality of catalyst beds 11a, 11b, 11c containing the one or more bifunctional catalysts as fixed bed catalyst or fixed bed catalysts, and which in the second reaction zone 12 has a catalyst bed 12a containing the one or more monofunctional catalysts as fixed bed catalyst or fixed bed catalysts. Further zones 14 can be provided and correspondingly equipped with a catalyst. Catalyst-free inert zones 13 are formed upstream of the first and downstream of the second reaction zone in the flow direction.

The diagrams shown in FIGS. 2A and 2B have already been explained above.

### BIBLIOGRAPHY

[1] Abass A. Olajire, “Valorization of greenhouse carbon dioxide emissions into value-added products by catalytic processes”, Journal of CO₂ Utilization 3-4 (2013)74-92.

[2] Ampelli C, Perathoner S, Centi G. 2015“CO₂ utilization: an enabling element to move to a resource- and energy-efficient chemical and fuel production”, Phil. Trans. R. Soc. A 2015 Vol. 373. http://dx.doi.org/10.1098/rsta.2014.0177.

[3 ] Erdogan Alper, Ozge Yuksel Orhan, “CO₂ utilization: Developments in conversion processes”, Petroleum 3 (2017) 109-126.

[4] R. Passalacqua, G. Centi, S. Perathoner, “Solar Production of Fuels from Water and CO₂: Perspectives and Opportunities for a Sustainable Use of Renewable Energy”, Oil & Gas Science and Technology—Rev. IFP Energies nouvelles, vol. 70 (2015), no. 5, pp. 799-815

[5] Gabriele Centi, Siglinda Perathoner, “Green Carbon Dioxide: Advances in CO₂ Utilization”, in particular “Chapter 1: Perspectives and State of the Art in Producing Solar Fuels and Chemicals from CO₂”, Wiley 2014.

[6] Elsje Alessandra Quadrelli, Gabriele Centi, Jean-Luc Duplan, Siglinda Perathoner, “Carbon Dioxide Recycling: Emerging Large-Scale Technologies with Industrial Potential”, ChemSusChem 2011, 4, 1194-1215.

[7] Ismaël Amghizar, Laurien A. Vanderwalle, Kevin M. Van Geem, Guy B. Marin, “New Trends in Olefin Production”, Engineering 3 (2017), 171-178.

[8] H. M. Torres Galvis, K. P. de Jong, 2013, “Catalysts for Production of Lower Olefins from Synthesis Gas: A Review”, ACS Catal., 3, 2130-2149.

[9] C. Wang, L. Xu, Q. Wang, “Review of Directly Producing Light Olefins via CO Hydrogenation”, Journal of Natural Gas Chemistry 2003, 12, 10-16.

[10] Haiyan Yang, Chen Zhang, Peng Gao, Hui Wang, Xiaopeng Li, Liangshu Zhong, Wei Wei, Yuhan Sun, “A review of the catalytic hydrogenation of carbon dioxide into value-added hydrocarbons”, Catal. Sci. Technol., 2017, 7, 4580-4598.

[11] Samrand Saeidi, Nor Aishah Saidina Amin, Mohammad Reza Rahimpour, “Hydrogenation of CO₂ to value-added products—A review and potential future developments”, Journal of CO₂ Utilization 5 (2014) 66-81.

[12] Gabriele Centi, Elsje Alessandra Quadrelli, Siglinda Perathoner, “Catalysis for CO₂ conversion: a key technology for rapid introduction of renewable energy in the value chain of chemical industries”, Energy Environ. Sci., 2013, 6, 1711-1731.

[13] G. A. Olah, A. Goeppert, G. K. S. Prakash, “Beyond Oil and Gas: The Methanol Economy”, Wiley-VCH 2006, chapter 13: “Methanol-Based Chemicals, Synthetic Hydrocarbons and Materials”, pp. 246-253.

[14] S. Xu, Y Zhi, J. Han, W. Zhang, X. Wu, T. Sun, Y. Wei, Z. Liu, “Chapter Two—Advances in Catalysis for Methanol-to-Olefins Conversion”, Advances in Catalysis, Academic Press, volume 61, 2017, pages 37-122, ISSN 0360-0564, ISBN 9780128120781.

[15] T. Mokrani, M. Scurell, “Gas Conversion to Liquid Fuels and Chemicals: The Methanol Route-Catalysis and Processes Development”, Catalysis Reviews Engineering (2009), 51(1), 1-145.

[16] J. Q. Chen, A. Bozzano, B. Glover, T. Fuglerud, S. Kvisle, “Recent advancements in ethylene and propylene production using the UOP/Hydro MTO process”, Catalysis Today 106 (2005), 103-107.

[17] J. Ott, V. Gronemann, F. Pontzen, E. Fiedler, G. Grossmann, D. B. Kersebohm, G. Weiss, C. Witte, “Methanol” in Ullmann's Encyclopedia of Industrial Chemistry (2012).

[18] K. Weissermel, H.-J. Arpe, Industrial Organic Chemistry (2003), 30-32, chapter “Methanol”.

[19] Unni Olsbye, “Single-Pass Catalytic Conversion of Syngas into Olefins via Methanol”, Angew. Chem. [Applied Chemistry] int. ed. 2016, 55, 7294-7295.

[20] K. Cheng, B. Gu, X. Liu, J. Kang, Q. Zhang, Y. Wang, “Direct and Highly Selective Conversion of Synthesis Gas into Lower Olefins: Design of a Bifunctional Catalyst Combining Methanol Synthesis and Carbon-Carbon Coupling”, Angew. Chem. [Applied Chemistry] int. ed. 2016, 55, 4725-4728.

[21] F. Jiao, J. Li, X. Pan, J. Xiao, H. Li, H. Ma, M. Wei, Y. Pan, Z. Zhou, M. Li, S. Miao, J. Li, Y. Zhu, D. Xiao, T. He, J. Yang, F. Qi, Q. Fu, X. Bao, “Selective conversion of syngas to light olefins,” Science 2016, 351, 1065-1068.

[22] R. Reimert, F. Marschner, H.-J. Renner, W. Boll, E. Supp, M. Brejc, W. Liebner, G. Schaub, “Gas Production, 2. Processes” in Ullmann's Encyclopedia of Industrial Chemistry (2012).

[23] P. Häussinger, R. Lohmüller, A. M. Watson, “Hydrogen, 2. Production” in Ullmann's Encyclopedia of Industrial Chemistry (2012).

[24] K. Weissermel, H.-J. Arpe, Industrial Organic Chemistry (2003), 15-24, chapter “Synthesis Gas”.

[25] D. San-José-Alonso, J. Juan-Juan, M. J. Illán-Gómez, M. C. Román-Martínez, Applied Catalysis A: General 371 (2009) 54-59.

[26] E. Schwab, A. Milanov, S. A. Schunk, A. Behrens, N. Schödel, Chem. Ing. Tech. (2015), 87, no. 4, 347-353.

[27] Wenhui Li, Haozhi Wang, Xiao Jiang, Jie Zhu, Zhongmin Liu, Xinwen Guo, Chunshan Song, “A short review of recent advances in CO₂ hydrogenation to hydrocarbons over heterogeneous catalysts”, RSC Adv., 2018, 8, 7651.

[28] Jiajian Gao, Chunmiao Jia, Bin Liu, “Direct and selective hydrogenation of CO₂ to ethylene and propene by bifunctional catalysts”, Catalysis Science & Technology, issue 23, 2017.

[29] Xiaoliang Liu, Mengheng Wang, Cheng Zhou, Wei Zhou, Kang Cheng, Jincan Kang, Qinghong Zhang, Weiping Deng, Ye Wang, “Selective transformation of carbon dioxide into lower olefins with a bifunctional catalyst composed of ZnGa₂O₄ and SAPO-34”, Chem. Commun., 2018, 54, 140-143.

[30] Jinjing Li, Xiulian Pan, Xinhe Bao, “Direct conversion of syngas into hydrocarbons over a core-shell Cr-Zn@SiO₂@SAPO-34 catalyst”, Chinese Journal of Catalysis 36 (2015) 1131-1135.

[31] Zelong Li, Jijie Wang, Yuanzhi Qu, Hailong Liu, Chizhou Tang, Shu Miao, Zhaochi Feng, Hongyu An, Can Li, “Highly Selective Conversion of Carbon Dioxide to Lower Olefins”, ACS Catal. 2017, 7, 8544-8548.

[32] Masahiro Fujiwara, Takahiro Satake, Kumi Shiokawa, Hiroaki Sakurai, “CO₂ hydrogenation for C2+ hydrocarbon synthesis over composite catalyst using surface modified HB zeolite”, Applied Catalysis B: Environmental 179 (2015) 37-43.

[33] Congming Li, Xingdong Yuan, Kaoru Fujimoto, “Direct synthesis of LPG from carbon dioxide over hybrid catalysts comprising modified methanol synthesis catalyst and (β-type zeolite”, Applied Catalysis A: General 475 (2014) 155-160.

[34] Peng Gao, Shanshan Dang, Shenggang Lia, Xianni Bu, Ziyu Liu, Minghuang Qiu, Chengguang Yang, Hui Wang, Liangshu Zhong, Yong Han, Qiang Liu, Wei Wei, Yuhan Sun, “Direct production of lower olefins from CO₂ conversion via bifunctional catalysis”, ACS Catal., 2018, 8 (1), pp 571-578.

[35] Peng Gao, Shenggang Li, Xianni Bu, Shanshan Dang, Ziyu Liu, Hui Wang, Liangshu Zhong, Minghuang Qiu, Chengguang Yang, Jun Cai, Wei Wei, Yuhan Sun, “Direct conversion of CO₂ into liquid fuels with high selectivity over a bifunctional catalyst”, Nature Chemistry, October 2017, 1-6.

[36] Young-Kwon Park, Kwang-Cheon Park, Son-Ki Ihm, “Hydrocarbon synthesis through CO₂ hydrogenation over CuZnOZrO₂/zeolite hybrid catalysts”, Catalysis Today 44 (1998) 165-173.

[37] Hania Ahouari, Ahcene Soualah, Anthony Le Valant, Ludovic Pinard, Yannick Pouilloux, “Hydrogenation of CO2 into hydrocarbons over bifunctional system Cu-ZnO/Al₂O₃+HZSM-5: Effect of proximity between the acidic and methanol synthesis sites”, C. R. Chimie 18 (2015) 1264-1269.

[38] Masahiro Fujiwara, Hiroaki Sakurai, Kumi Shiokawa, Yasuo Iizuka, “Synthesis of C2+ hydrocarbons by CO₂ hydrogenation over the composite catalyst of Cu-Zn-Al oxide and HB zeolite using two-stage reactor system under low pressure”, Catalysis Today 242 (2015) 255-260.

[39] Congming Li, Kaoru Fujimoto, “Efficient conversion of carbon dioxide to non-methane light hydrocarbons—Two stage process with intercooler”, Fuel Processing Technology 136 (2015) 50-55.

[40] G. Emig, E. Klemm, “Technische Chemie: Einführung in die chemische Reaktionstechnik (Springer textbook)”, [Technical Chemistry: Introduction to Chemical Reaction Technology], 5th edition, 2006.

[41] M. Baerns, A. Behr, A. Brehm, J. Gmehling, K.-O. Hinrichsen, H. Hofmann, U. Onken, R. Palkovits, A. Renken, “Technische Chemie” [Technical Chemistry], 2nd edition, Wiley-VCH, 2014.

1. A method for producing one or more hydrocarbons, comprising:
feeding a process gas stream to a reactor arrangement, the process gas stream comprising carbon dioxide and/or carbon monoxide with hydrogen;
converting, within the reactor arrangement, the process gas stream at least in part in a first reaction step into one or more oxygenates, which pass into the process gas stream;
converting, within the reactor arrangement, the one or more oxygenates in the process gas stream at least in part in a second reaction step into one or more hydrocarbons, which pass into the process gas stream;
wherein:
the process gas stream conducted in a flow direction through the reactor arrangement;
the reactor arrangement has one or more reactors, the one or more reactors comprising a first reaction zone and a second reaction zone, wherein the second reaction zone is arranged downstream of the first reaction zone in the flow direction; and
the first reaction zone and the second reaction zone are equipped with catalysts in such a way that:
the first and second reaction steps are catalyzed in the first reaction zone;
the second reaction step is catalyzed in the second reaction zone; and
in the second reaction zone, the first reaction step is not catalyzed or is catalyzed to a lesser extent than in the first reaction zone.

2. The method according to claim 1, in which the first reaction zone is equipped with one or more first catalysts, which catalyze the first reaction step, and also with one or more second catalysts, which catalyze the second reaction step.

3. The method according to claim 1, in which the first reaction zone is equipped with one or more bifunctional catalysts, which catalyze the first and second reaction steps.

4. The method according to claim 1, in which catalyst fixed beds are used in the first reaction zone and in the second reaction zone.

5. The method according to claim 1, in which the reactor or the reactors is or are designed as a tube bundle reactor, which is or are cooled using a cooling medium which is conducted in cocurrent or countercurrent flow with the process gas stream.

6. The method according to claim 1, in which the first reaction zone has catalyst beds which are arranged one behind the other in the flow direction and have a plurality of different catalysts or a catalyst with different activities.

7. The method according to claim 1, in which one or more catalyst-free inert zones are formed.

8. The method according to claim 1, in which upstream of the first reaction zone, one or more further reaction zones are arranged, which contain one or more catalysts, which catalyze at least one further reaction, in particular a water-gas shift reaction and/or the formation of methanol and/or dimethyl ether as an intermediate.

9. The method according to claim 1, which is performed at a pressure level of 10 to 100 bar and a temperature level of 150 to 580° C.

10. The method according to claim 1, in which the process gas stream of the reactor arrangement is fed with a stoichiometric module of 1.5 to 10.

11. The method according to claim 1, in which the one or more oxygenates comprise methanol and/or dimethyl ether, and in which the one or more hydrocarbons comprise ethylene and/or propylene.

12. The method according to claim 1, in which the process gas stream has further components, in particular methane and/or higher hydrocarbons.

13. The method according to claim 1, in which at least the hydrocarbons are at least partially separated off from the process gas stream after passage through the reactor arrangement, wherein a remaining residue of the process gas stream is at least partially returned to the inlet of the reactor arrangement.

14. A system for producing a target compound, which is configured to convert hydrogen with carbon dioxide and/or carbon monoxide in a process gas stream, the system comprising a reactor arrangement comprising one or more reactors comprising a first reaction zone and a second reaction zone, the second reaction zone being arranged downstream of the firs reaction zone in a flow direction;
wherein:
the process gas stream is converted, at least in part, in a first reaction step into one or more oxygenates, which pass into the process gas stream;
the one or more oxygenates in the process gas stream is/are converted, at least in part, in a second reaction step into one or more hydrocarbons, which pass into the process gas stream;
the system is configured to conduct the process gas stream in the flow direction through the reactor arrangement; and
the first reaction zone and the second reaction zone are equipped with catalysts in such a way that:
the first and second reaction steps are catalyzed in the first reaction zone;
the second reaction step is catalyzed in the second reaction zones; and
in the second reaction zone, the first reaction step is not catalyzed or is catalyzed to a lesser extent than in the first reaction zone.

15. The system according to claim 14, which is configured to perform a method according to claim 1.

16. The method according to claim 10, in which the process gas stream of the reactor arrangement is fed with a stoichiometric module of 2 to 4.

17. The method according to claim 9, wherein the method is performed at a pressure level of 12 to 50 bar and a temperature level of 200 to 450° C.

18. The method according to claim 17, wherein the method is performed at a pressure level of 15 to 35 bar and a temperature level of 250 to 400° C.

19. The method according to claim 18, in which the process gas stream of the reactor arrangement is fed with a stoichiometric module of 1.5 to 10.

20. The method according to claim 9, in which the process gas stream of the reactor arrangement is fed with a stoichiometric module of 1.5 to 10.
